(19) United States
(12) Patent Application Publication
(10) Pub. No.: US 2018/0236133 A1
(43) Pub. Date: Aug. 23, 2018

(54) METHODS OF PRODUCING EXTRACELLULAR MATRIX USEABLE IN BREAST IMPLANT SURGERY AND OTHER MATRIX PRODUCTION

(71) Applicant: DermaGenesis LLC, Pompano Beach, FL (US)

(72) Inventors: David Vogel, Pompano Beach, FL (US); Richard Vogel, Pompano Beach, FL (US); Derek Cox, Pompano Beach, FL (US); Jessica Bills, Pompano Beach, FL (US)

(21) Appl. No.: 15/902,270

(22) Filed: Feb. 22, 2018

Related U.S. Application Data
(60) Provisional application No. 62/462,067, filed on Feb. 22, 2017.

(51) Int. Cl. A61L 27/36; A61L 27/24; A61L 27/20

(52) U.S. Cl. CPC A61L 27/3633; A61L 2430/04; A61L 27/20; A61L 27/24

(57) ABSTRACT

Methodologies in which GAG/collagen mixtures are homogenized into slurries, lyophilized, and dried, have been producing solid matrix products that have favorable tackiness for use in breast reconstruction surgery.

### FIELD OF THE INVENTION

The invention relates to production of matrix product, more particularly, to extracellular matrix (ECM) product useable in breast implant surgery.

### BACKGROUND OF THE INVENTION

In breast reconstruction surgery, silicone materials have been conventionally used but silicone has slippery characteristics, and slipperiness of materials being used in breast reconstruction surgery is a disadvantage. Some work has been done on silicone-based materials to try to reduce the slipperiness. Other work has been done in directions of exploring non-silicone materials to use in breast reconstruction surgery.

In the direction of textured exterior surfaces for breast implants, the patent literature includes the following:

U.S. Pat. No. 6,692,527 for “Non-rotating breast implant,” by Bellin, et al., issued Feb. 17, 2004.

U.S. Pat. No. 7,105,116 for “Non-rotating breast implant,” by Bellin, et al., issued Sep. 12, 2006.

U.S. Pat. No. 8,043,373 for “All-barrier elastomeric gel-filled breast prosthesis,” by Schuessler, et al., issued Oct. 25, 2011 to Allergan, Inc.

U.S. Pat. Pub. 20120101574 for “Implantable materials,” by Goraltchouk, et al. (Allergan), published Apr. 26, 2012.

U.S. Pat. No. 8,197,542 for “Self supporting implant in a human body and method for making the same without capsular contracture,” by Becker, issued Jun. 12, 2012.

U.S. Pat. No. 8,313,527 for “Soft prosthesis shell texturing method,” by Powell, et al., issued Nov. 20, 2012 to Allergan, Inc.

U.S. Pat. Pub. 20140180412 for “Device and method for making a variable surface breast implant,” by Nieto, et al. (Allergan), published Jun. 26, 2014.

U.S. Pat. Pub. 20140242258 for “Implantable materials,” by Goraltchouk, et al. (Allergan), published Aug. 28, 2014.

U.S. Pat. No. 9,138,310 for “Soft prosthesis shell texturing method,” by Powell, et al., issued Sep. 22, 2015, to Allergan, Inc.

U.S. Pat. Pub. 20140305854 for “Variable surface breast implant,” by Schuessler, et al. (Allergan), published Oct. 29, 2015.

U.S. Pat. Pub. 20150327986 for “Textured breast implant and methods of making same,” by Nieto, et al. (Allergan), published Nov. 19, 2015.

U.S. Pat. No. 9,688,006 for “Device and method for making a variable surface breast implant,” by Nieto, et al. (Allergan), issued Jun. 27, 2017 to Allergan, Inc.

U.S. Pat. Pub. 20170290652 for “Device and method for making a variable surface breast implant,” by Nieto, et al. (Allergan), published Oct. 12, 2017.

Some uses of certain ECM products have been described in the literature for breast reconstruction products. See. e.g., Matheny et al., “Breast implants and compositions of extracellular matrix,” US 2008/0281419, published Nov. 13, 2008; Matheny, “Breast implants and compositions of extracellular matrix,” US 2010/0010627, published Jan. 14, 2010; Ward, et al., “Tissue scaffolds derived from forestomach extracellular matrix,” US 20140335144, published Nov. 13, 2014; Codori-Hurff, et al., “Mastopexy and breast reconstruction prostheses and method,” US 20120158134, published Jun. 21, 2012; Badylak, et al., “Extracellular matrix mesh coating,” US 20150297798, published Oct. 22, 2015.

There remain unmet needs for non-slippery products suitable for use in breast reconstruction surgery.

### SUMMARY OF THE INVENTION

The invention addresses the aspect of the prior art that thus far, conventional ECM has been relatively low in tackiness, and generally lacking in self-cohesiveness, and more along the lines of being characterized by slipperiness. An objective of the invention is to produce ECM useable in surgery (especially breast implant surgery) and having improved tackiness compared to conventional ECM product.

The invention in a preferred embodiment provides a method of producing tacky ECM product, comprising steps of: mixing glycosaminoglycans (GAGs) and collagen (such as, e.g., micronized collagen) to form a collagen mixture (such as, e.g., a mixing step that comprises adding GAGs into a container having collagen therein to form the collagen mixture); followed by homogenizing the collagen mixture to form a slurry; lyophilizing the slurry until the slurry is completely frozen; followed by after the slurry is completely frozen, drying the frozen slurry to form a matrix, such as, e.g., inventive methods further comprising, before the adding of GAGs into the container having collagen therein, performing a step of introducing a mass of collagen into the container (such as, e.g., a container of acetic acid (ethanoic acid); a container of hydrochloric acid (HCl; muriatic acid); a container of formic acid (methanoic acid); a container of propanoic acid (propionic acid); etc.); inventive methods wherein the step of adding GAGs comprises adding GAGs drop-wise; inventive methods further comprising, after homogenizing the collagen mixture to form the slurry, performing a step of degassing the slurry before lyophilizing the slurry; inventive methods wherein the GAGs comprise Hyaluronic acid (HA/HyA); inventive methods wherein the GAGs comprise Chondroitin-6-sulfate (C6S); inventive methods wherein the GAGs comprise Hyaluronic acid and C6S; inventive methods wherein the GAGs comprise one or more of selected from the group consisting of: Hyaluronic acid (HA): Chondroitin-6-sulfate (C6S); Keratin sulfate; Heparin; Heparin sulfate; and Fibronectin; inventive methods wherein the drying step comprises activating a vacuum, wherein the vacuum activation is performed only after the slurry is completely frozen; inventive methods wherein during the drying step, the slurry is disposed on a tray (such as, e.g., methods wherein during the drying step, the tray temperature is in a range of −45 to −3° C.; methods wherein during the drying step, no part of the slurry is permitted to reach or exceed 0° C.; etc.), and other inventive methods.

In another preferred embodiment, the invention provides a surgically-implantable product, comprising an ECM that has a tackiness that is self-cohesive, preferably an ECM that comprises collagen (such as, e.g., bovine collagen, etc.), an ECM that comprises an envelope shape, etc.

### BRIEF DESCRIPTION OF THE DRAWINGS

FIG. 1 is a diagram of method steps according to an embodiment of the invention.

### DESCRIPTION OF A PREFERRED EMBODIMENT OF THE INVENTION

The invention provides surgically-useable, relatively-tacky ECM product (such as, e.g., ECM product produced from collagen (such as, e.g., bovine collagen)), and methods (such as the methodology of Inventive Examples 1-2M1 hereinbelow) for producing such relatively-tacky ECM product.

Preferably the ECM is sufficiently tacky to be self-cohesive.

Tacky ECM product (especially self-cohesive ECM) according to the invention is useable in certain surgeries, especially in breast reconstruction surgery, where less-slippery ECM product is advantageous.

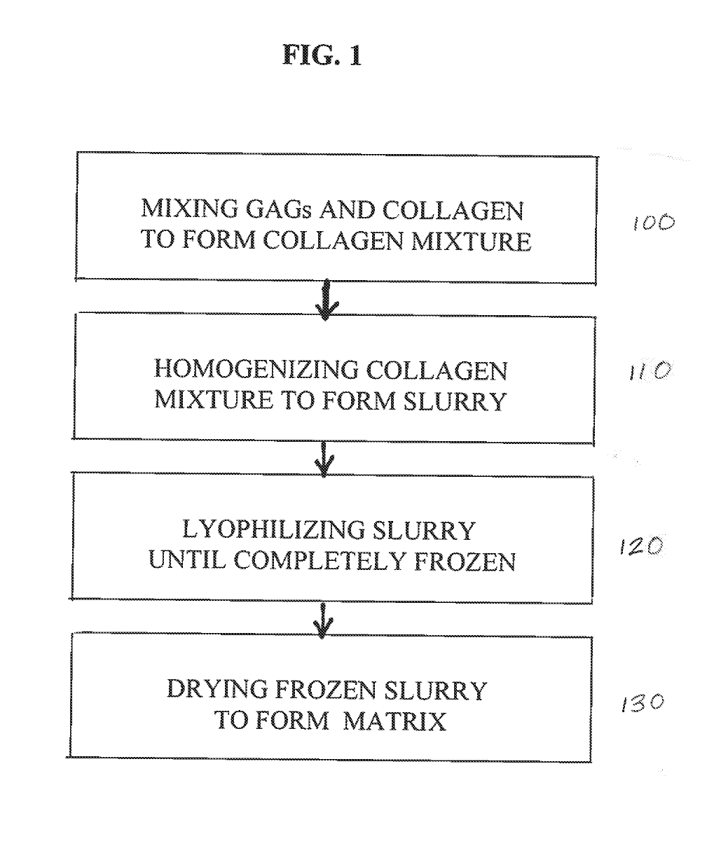

As may be appreciated with reference to FIG. 1, a preferred method for making relatively-tacky ECM product comprises steps of mixing 100 glycosaminoglycans (GAGs) and collagen to form a collagen mixture; homogenizing 110 the collagen mixture to form a slurry; lyophilizing 120 the slurry until completely frozen; and drying 130 the frozen slurry to form a matrix.

The following examples are by way of illustration, and are not intended to be limiting.

### Inventive Example 1 (Production of 125 ml Batch)

### Materials

DDW [H₂O (aq.)]—(dry distilled water/deionized H₂O)

Glacial acetic acid

Decellularized, lyophilized micronized bovine type I collagen

Glycosaminoglycans (GAGs)
Hyaluronic acid (HA/HyA)
Chondroitin-6-sulfate (C6S)

Homogenizer with cooling apparatus

Cooling apparatus
Alcohol (ethanol/isopropanol [isopropyl alcohol])
Dry ice [CO₂(s.)]—(solid carbon dioxide)

Lyophilizer and vacuum pump

Beakers and flasks

### Procedure

### 1. Preparation of Stock Solutions

a. Acetic acid (0.05M dilution)
   i. Introduce about 365 mg glacial acetic acid to a beaker.
   ii. Add 125 mL DDW and stir.
   iii. Remove 25 mL of the stock acetic acid for titration of GAGs.
   iv. Store the remainder of the stock acetic acid (˜100 mL) for later use.
b. Glycosaminoglycans (GAG; 0.02% wt/vol)
   i. Add between 1-25 mg of GAGs (chondroitin-6-sulfate/hyaluronic acid) to a beaker and dilute with 25 mL of the stock acetic acid solution generated in the last step.
   ii. Stir until homogenous (ex. magnetic stir bar) and set aside. The GAG stock solution will be added to the collagen slurry later via drop-wise addition.

### 2. Preparation of Bovine Collagen

a. Collect the homogenization vessel and introduce micronized bovine collagen to the container, followed by the stock acetic acid and let the tissue soak for a few minutes.
b. Introduce 0.25-1.5 g bovine collagen to the container. Immerse the mass of bovine collagen in ˜100 mL of the stock acetic acid solution and let the tissue soak for a few minutes. (NOTE: This step should be equivalent to adding the 80% remaining proportion of the 0.05M acetic acid solution to the micronized bovine collagen.)
c. Add 25 mL of GAG (in acetic acid solution) drop-wise.

### 3. Homogenization of Collagen Matrix

a. Preparation of the homogenization equipment: Place the homogenization vessel in the heat sink and let the temperature of the inside of the vessel decrease.
b. Introduce the homogenizer to vessel by lowering the device into the mixture, submerging the head (where the blades are located) and an additional ˜2 cm of the stem in the slurry.
c. Stir the mixture aggressively for about an hour or two.

### 4. Degassing the Slurry

a. Degassing is essential for optimal matrix formation and organization, and can be achieved by introducing the slurry to a vacuum. One method of achieving this vacuum consists of filling a vacuum-enabled Erlenmeyer flask with slurry, stoppering the top, and pulling vacuum on the contents of the flask.
b. 15-30 minutes of degassing should be sufficient.
c. During the degassing process, initiate the lyophilizer and set the tray temperature to −45° C. (228K) to allow the system to reach equilibrium.

### 5. Lyophilization of Slurry

a. Preparation
   i. Transfer degassed and homogenized slurry to lyophilization tray.
   ii. Once the lyophilizer has acclimated itself to the tray temperature (˜1 hour), introduce the tray to the machine.
b. Freezing: The slurry MUST freeze completely before beginning the drying process.
c. Drying
   i. When the slurry is completely frozen, the vacuum, can be activated.
   ii. Atmospheric pressure in the lyophilization chamber will decrease until the pressure in the system reaches maximum vacuum.
   iii. At this point, set the tray temperature to below the freezing temperature of water to encourage the sublimation of H₂O. (NOTE1: As freezing, there is no concern of “over-drying.” Therefore it is better to dry for more time, rather than losing a batch due to insufficient drying. This is important, because drying time will be affected by factors such as tray temperature during drying, and also the thickness of the matrix.) (NOTE2: While the tray temperature can be any value from −45° C. to −3 C, it is important not to let any volume of the slurry reach 0° C. or above. If any H₂O is allowed to convert to liquid phase, the entire matrix will have to be refrozen and dried again before sterilization.)
d. Removal of lyophilized slurry
   i. Set the tray temperature to a value representing room temperature.
   ii. Once the tray has acclimated to the new temperature settings and reaches equilibrium, the vacuum can be disconnected and the lyophilization chamber can be allowed to return to STP (standard temperature and pressure).
   iii. Once STP has been achieved, the door can be opened and the lyophilized collagen can be removed. (NOTE: Due to negative pressure, the door will be impossible to open until STP is achieved.)

### 6. Treatment:

Matrices should be cut to appropriate product sizes (if necessary) and packaged.

### 7. Sterilization

### Inventive Example 2

### Materials

Beakers and flasks

DDW [H₂O (aq.)]—(dry distilled water/deionized H₂O)

Glacial acetic acid [C₂H₄O₂ (aq.)]

Decellularized, lyophilized micronized bovine type I collagen (s.)

Glycosaminoglycans (GAGs)
Chondroitin-6-sulfate [C6S (s.)]
Hyaluronic acid [HA/HyA (s.)]

Industrial mixer

Cooling apparatus

Vacuum pump

Lyophilizer

### Procedure

### 1. Preparation of Slurry

a. NOTE: The volume of H₂O is 0.3% less than the volume of slurry in its mixed state. This water content will sublimate during lyophilization and leave the final product. Therefore, this Example 2 sets forth instructions as if the volumes are equivalent. An example, in the case of a 750 mL slurry, is that hereinbelow in Example 2 750 g (750 mL) of H₂O is set forth instead of 747.75 g (747.75 mL).
b. Dilution of acetic acid (0.05M dilution)
   i. To any volume (mL) of DDW, add mass (mg) of glacial acetic acid, preferably in the proportion set out in see Example 2A, and stir until mixed.
   ii. Set aside for preparation of the collagen slurry.
c. Preparation of collagen (05% wt/vol)
   i. Collect the homogenization vessel and introduce the micronized collagen to the container, preferably in the proportion set out in Example 2C.
d. Preparation of glycosaminoglycans (GAG; 1.01% wt/vol)
   i. To the same homogenization vessel, add mass (mg) of chrondroitin-6-sulfate, preferably according to the relationship set out in Example 2E.
   ii. In addition, introduce mass (mg) of hyaluronic acid, preferably according to Example 2F.

### 2. Homogenization of Slurry

a. Preparation of the cooling system and homogenization unit.
Assemble the homogenization unit above the cooling system (preferred examples of cooling systems are given in Ex. 2H and Ex. 2H1), so that it may be, at a future time, lowered into the homogenization vessel after being placed in the bath.
Activate the cooling system and allow equilibrium to be reached before mixing.

b. Production of the slurry
i. Add the diluted acetic acid solution to the homogenization vessel and place the homogenization vessel in the bath.
   1. Introduce homogenizer head into vessel by lowering the device into the mixture, submerging the head (where the blades are located) and an additional −2 cm of the stem in the slurry.
   2. Let the temperature of the inside of the vessel (the slurry) reach equilibrium with the bath.
   3. To stir the slurry without adding heat energy to the solution, use the lowest setting on the homogenizer while the cooling system reaches equilibrium.
ii. When the cooling apparatus is confirmed to be withdrawing heat from the system, the homogenization process can be initiated.

c. Agitation of the mixture: The mixture is agitated at a minimum rate of 10,000 rpm (maximum 24,000 rpm) for up to 2 hours. The purpose of this agitation step is to achieve complete homogenization. Balancing is wanted in this step between temperature of the slurry, time spent homogenizing, and the rate of the homogenization unit. This step's objective is complete homogenization, such that the slurry appears completely uniform and without particulate matter.

d. Degassing of the slurry: Degassing is essential for optimal matrix formation and organization, and can be achieved by introducing the slurry to a vacuum. One hour of degassing is sufficient. The purpose of the vacuum is to remove all the trapped gas within the liquid matrix and to essentially purify the slurry by removing air.

3. Lyophilization of slurry
a. Preparation
   i. Transfer degassed and homogenized slurry to lyophilization tray, being careful to limit atmospheric exposure. The more erratic the dispensing, the more air will re-enter the slurry, which is unwanted. Take care to either dispense using machinery or to pour delicately.
   ii. Once the lyophilizer has acclimated itself to the tray temperature (˜1 hour), introduce the tray to the machine.
b. Freezing: The slurry MUST freeze completely before the drying process can begin. The slurry can freeze indefinitely and without issue; therefore it is essential that no liquid remains. Put another way, the vacuum should not be initiated until the slurry is totally frozen.
c. Drying: When the slurry is completely frozen, the vacuum can be activated. As with freezing, there is no concern of “over-drying.” Therefore, it is better to dry for more time, rather than losing a batch due to insufficient drying. This important, because drying time will be affected by factors such as tray temperature during drying, and also thickness of the matrix.
d. Removal of lyophilized slurry.

### Inventive Example 2A

The preferred relationship between the desired volume of slurry in milliliters (mL) and mass of glacial acetic acid in milligrams (mg) is:

3*desired volume (mL)_{slurry}=mass (mg)_{acetic acid}

### Inventive Example 2B (750 mL Slurry)

An example of dilution of acetic acid during slurry preparation in Example 2 is:

1. Introduce 2.250 mg (2.250 g; ˜2.154 mL) glacial acetic acid to a 1 L beaker.
2. Add 750 mL (750 g) DDW and stir.
3. Set aside for later use.

### Inventive Example 2C

The preferred relationship between the desired volume of slurry in milliliters (mL) and mass of micronized collagen in grams (g) is:

$$\frac{desired {volume ( mL )}_{slurry}}{200} = mass {( g )}_{collagen}$$

### Inventive Example 2D (750 mL Slurry)

In an example according to Ex. 2C, 3.75 g collagen is introduced to the container slurry preparation according to Ex. 2B.

### Inventive Example 2E

The preferred relationship between the desired volume of slurry in milliliters (mL) and mass of chondroitin-6-sulfate (C6S) in milligrams (mg) is:

$$\frac{desired {volume ( mL )}_{slurry}}{10} = mass {( g )}_{C 6 S}$$

### Inventive Example 2F

The preferred relationship between the desired volume of slurry in milliliters (mL) and mass of hyaluronic acid (HA) in milligrams (mg) is:

10*desired volume (mL)_{slurry}=mass (mg)_{HA}

### Inventive Example 2G

An example of slurry-preparation according to Exs. 2, 2F and 2G is as follows:
1. Add 75 mg (0.075 g) of chondroitin-6-sulfate (C6S) to the homogenization vessel that contains the bovine collagen.
2. Add 7500 mg (7.50 g) of hyaluronic acid (HA) to the homogenization vessel that contains the bovine collagen and the C6S.

### Inventive Example 2H

In the slurry-homogenization step of Example 2, for preparation of the cooling system and homogenization, cold rooms are one of the best ways to keep the temperature of the slurry at an acceptable level (18° C. or lower).

### Inventive Example 2H1

In the slurry-homogenization step of Example 2, for preparation of the cooling system and homogenization, an option for a sterile endothermic system consists of the vessel being introduced to a liquid bath, or homogenized inside a refrigeration unit. The bath must be able to remain liquid at cold temperatures, so water or propylene glycol is recommended as the solvent. By adding to the bath a coolant, an example of which is CO, (s.) [carbon dioxide, solid (dry ice)], the temperature of the heat sink can be kept as close as possible to 0° C. (273K).

### Inventive Example 2I

For the agitation step in Example 2, agitation that has been found to be efficient (for the purpose of carrying out complete homogenization, such that the slurry appears completely uniform and without particulate matter) is homogenization at 15,000 rpm (or the max rate without overheating the slurry) for 30 to 60 minutes, and including a few short bursts of higher rates either throughout or at the end.

### Inventive Example 2J (Introducing Slurry to a Vacuum)

One method of achieving a vacuum in a step of introducing a slurry to a vacuum is to fill a vacuum-enabled Erlenmeyer flask with slurry, stopper the top, and pull vacuum on the contents of the flask.

### Inventive Example 2J1 (Degassing)

Bubbles only appear in a slurry if gas is still trapped within the mix; this is a key indicator that the slurry contains gas, and that more degassing is needed. The longer the slurry is exposed to negative pressure, the less gas volume will exist within the slurry; therefore, as the degassing process approaches completion, the bubbles will become smaller and less numerous.

### Inventive Example 2J2

An indication of a fully purged slurry is lack of bubbles when exposed to the vacuum. Lack of bubbles in the slurry is used as an endpoint for the degassing step.

### Inventive Example 2K

During the degassing process, preferably the lyophilizer is activated, and the tray temperature is set to −25° C. (228K), and the system is allowed to reach equilibrium.

### Inventive Example 2K1

Because the degassing step takes approximately 1 hour, it is optimal, during the degassing of the slurry, to begin a lyophilization acclimation process.

### Inventive Example 2L (Drying)

In the drying step, after the vacuum is activated, atmospheric pressure in the lyophilization chamber will decrease until the pressure in the system reaches maximum vacuum.

An atmospheric pressure value of 200 mtorr (0.00026 atm=0.00026 barr=26.67 Pa=0.00387 PSI) is sufficient. A stronger vacuum (less pressure) will increase efficiency.

### Inventive Example 2L1 (Drying)

In the drying step, at a point where the pressure in the system reaches maximum vacuum, set the tray temperature to −3° C. (270K) to encourage the sublimation of H₂O.

NOTE: While the tray temperature can thusly be increased from −25° C. to −3° C., it is important not to let any volume of the slurry reach 0° C. or above. If any H₂O is allowed to convert to liquid phase, the entire matrix will have to be refrozen and dried again before sterilization.

### Inventive Example 2M (Removal of Lyophilized Slurry)

An example of removal of lyophilized slurry from a tray is as follows:
i. Set the tray temperature to a value representing room temperature (18-21° C.).
ii. Once tray has acclimated to the new temperature setting and reaches equilibrium, the vacuum can be disconnected and the lyophilization chamber can be allowed to return to STP (standard temperature and pressure).
iii. Once STP has been achieved, the door can be opened and the lyophilized matrix can be removed. If any liquid phase slurry remains, the entire matrix must be refrozen and dried again before sterilization.
   NOTE: Due to negative pressure, the door will be impossible to open until STP is achieved.

### Inventive Example 2M1

After removal of the lyophilized slurry, the solid is cut to appropriate product sizes (if necessary) and packaged.

### Inventive Example 2N

A degassed slurry was produced according to Example 2. The degassed slurry was pipetted into a petri dish, and took the form of its container. A uniform consistency and lack of gas bubbles within the matrix were observed, which suggested that a high quality matrix would form during lyophilization.

### Inventive Example 2N1

Example 2N's degassed slurry in petri dish was subjected to lyophilization, and a dry matrix was produced. The dry matrix kept its shape and was easily removed from the petri dish using tweezers.

While the matrix keeps its shape, meanwhile it is also extremely porous and flexible, like a sponge.

### Inventive Example 2O

A degassed slurry is produced according to Example 2. The degassed slurry is dispensed (such as, e.g., by pipetting; by pouring; by dispensing; etc.) into or onto a container (such as, e.g., a petri dish; a tray (preferably a metal tray); a mold; etc.).

### Inventive Example 3

In this example, a collagen-containing matrix useable as a breast implant has lower slipperiness (i.e., higher tackiness) than silicone implants sold by Allergan (Natrelle), Mentor, and Sientra.

### Inventive Example 4 (Envelope Shape)

A matrix is constructed according to an Example above (or a scaled-up variation thereof), in an envelope shape comprising a first open edge and a second open edge.

### Inventive Example 4A

In this example, the matrix of Example 4 has a thickness of about 1 mm to 20 mm.

### Inventive Example 5

Into the envelope of Example 4 is placed a breast implant product (such as, e.g., a slippery implant, a textured implant, etc.) after which placement the first open edge is brought into contact with the second open edge, and sealing closure is manually accomplished by pressing two fingertips against each other with the envelope edges between the two pressed-together fingertips.

### Inventive Example 5A

In this example according to Example 5, the volume of the implant product inside the envelope is in a range of about 1 mL to 500 mL, and the surface area defined by pressed-together edges is in a range of about 10 cm² to 150 cm².

### Inventive Example 5B

In this example according to Example 5, a 300 cc breast implant is placed inside the matrix envelope, and the surface area defined by pressed-together edges is in a range of about 10 cm² to 150 cm².

### Inventive Example 6

A matrix sample that had been made by the inventors was subjected to “closure” experimentation. Pressures in a range of 18-998 grams applied for under 2 seconds were found to bring about closure. That a pressure as low as 100 grams was sufficient to accomplish closure is considered a unique and important characteristic of the inventive matrix; that closure was accomplished with even lower pressures, and as low as 18 grams of pressure, is considered to be a highly advantageous characteristic of the material.

The above described embodiments are set forth by way of example and are not limiting. It will be readily apparent that obvious modifications, derivations and variations can be made to the embodiments. Accordingly, the claims appended hereto should be read in their full scope including any such modifications, derivations and variations.

What we claim as our invention is as follows:

1. A method of producing tacky ECM product, comprising steps of:
mixing glycosaminoglycans (GAGs) and collagen to form a collagen mixture; followed by
homogenizing the collagen mixture to form a slurry;
lyophilizing the slurry until the slurry is completely frozen; followed by
after the slurry is completely frozen, drying the frozen slurry to form a matrix.

2. The method of claim 1, wherein the step of mixing GAGs and collagen to form a collagen mixture comprises adding GAGs into a container having collagen therein to form the collagen mixture.

3. The method of claim 2, further comprising, before the adding of GAGs into the container having collagen therein, performing a step of introducing a mass of collagen into the container.

4. The method of claim 3, wherein the container into which the mass of collagen is introduced is a container of acetic acid.

5. The method of claim 3, wherein the container into which the mass of collagen is introduced is a container of acid selected from the group consisting of:
acetic acid (ethanoic acid);
hydrochloric acid (HCl; muriatic acid);
formic acid (methanoic acid); and
propanoic acid (propionic acid).

6. The method of claim 1, wherein the step of adding GAGs comprises adding GAGs drop-wise.

7. The method of claim 1, further comprising, after homogenizing the collagen mixture to form the slurry, performing a step of degassing the slurry before lyophilizing the slurry.

8. The method of claim 1, wherein the GAGs comprise Hyaluronic acid (HA/HyA).

9. The method of claim 1, wherein the GAGs comprise Chondroitin-6-sulfate (C6S).

10. The method of claim 1, wherein the GAGs comprise Hyaluronic acid and C6S.

11. The method of claim 1, wherein the GAGs comprise one or more of selected from the group consisting of:
Hyaluronic acid (HA);
Chondroitin-6-sulfate (C6S);
Keratin sulfate;
Heparin;
Heparin sulfate; and
Fibronectin.

12. The method of claim 1, wherein the collagen being introduced into the collagen is micronized collagen.

13. The method of claim 1, wherein the drying step comprises activating a vacuum, wherein the vacuum activation is performed only after the slurry is completely frozen.

14. The method of claim 1, wherein during the drying step, the slurry is disposed on a tray.

15. The method of claim 14, wherein during the drying step, the tray temperature is in a range of −45 to −3° C.

16. The method of claim 14, wherein during the drying step, no part of the slurry is permitted to reach or exceed 0° C.

17. A surgically-implantable product, comprising an ECM that has a tackiness that is self-cohesive.

18. The product of claim 17, comprising collagen.

19. The product of claim 17, comprising bovine collagen.

20. The product of claim 17, wherein the ECM comprises an envelope shape.
